(19) United States
(12) Patent Application Publication
(10) Pub. No.: US 2004/0243010 A1
(43) Pub. Date: Dec. 2, 2004

(54) Use of BNP during stress testing for the detection and risk stratification of individuals with suspected coronary artery disease

(76) Inventors: William A. Zoghbi, Houston, TX (US); Htut K. Win, Pearland, TX (US)

Correspondence Address: FULBRIGHT & JAWORSKI, LLP, 1301 MCKINNEY, SUITE 5100, HOUSTON, TX, 77010-3095, US

(21) Appl. No.: 10/804,668

(22) Filed: Mar. 19, 2004

Related U.S. Application Data
(60) Provisional application No. 60/455,928, filed on Mar. 19, 2003.

(51) Int. Cl. A61B 5/02

(52) U.S. Cl. 600/508

(57) ABSTRACT

A method of early detection and diagnosis of coronary artery disease in a patient comprising a) inducing cardiac stress in the patient by either exercise or pharmacological agents; and b) measuring the patient's BNP level before and after said cardiac stress. Additionally, there is provided a method of risk stratification of severity of coronary artery disease.

### CROSS-REFERENCE TO RELATED APPLICATIONS

This application claims the benefit of U.S. Provisional Application No. 60/455,928, filed Mar. 19, 2003, which is hereby incorporated by reference in its entirety.

### TECHNICAL FIELD

The present invention relates to methods, compositions, and devices for the measurement of BNP during stress, and more particularly to the use of such measurement as a predictor in the diagnosis, prognosis, and treatment of patients with suspected or known coronary artery disease.

### BACKGROUND OF THE INVENTION

B-type natriuretic peptide (BNP) is a 32-amino acid neurohormone that is stored in and secreted predominantly from membrane granules in the heart ventricles, and is continuously released from the heart in response to both ventricle volume expansion and overload. The functions of BNP include natriuresis, vasodilation, inhibition of the renin-angiotensin-aldosterone axis, and inhibition of sympathetic nerve activity.

The precursor to BNP is synthesized as an 108-amino acid molecule referred to as “pre pro BNP”, which is proteolytically cleaved into a 76-amino acid molecule known as NT pro BNP, and the 32-amino acid BNP.

The blood level of BNP compares well with left ventricular function expressed as ejection fraction. BNP is currently used as a marker of left ventricular dysfunction. It also seems to provide independent prognostic information regarding survival, and left ventricular remodeling after myocardial infarction (Stein et. al., 1998).

BNP levels are also raised in patients with acute myocardial infarction (MI) and regional wall motion abnormality. Following acute MI, BNP levels rise over the first 24 hours, and then stabilize. (Omland et. al., 1996) A single measurement of BNP, obtained within the first few days after the onset of ischemic symptoms, provides predictive information for use in risk stratification in acute coronary syndromes. (De Lemos et. al., 2001) Acute coronary syndromes consist of unstable angina pectoris, non-ST-segment-elevation myocardial infarction, and ST-segment-elevation myocardial infarction. Acute coronary syndromes are the result of arterial plaque disruption or endothelial damage (without plaque rupture) with resultant mural thrombus formation. Baseline BNP and N-terminal BNP levels (or NT proBNP) vary among individuals and are affected by factors such as age and sex, and thus absolute, nontemporary increases in BNP levels have limited usefulness as indicators of reversible ischemia.

BNP levels do not rise in exercise in subjects with normal cardiac function. However, BNP levels rise during exercise in patients after myocardial infarction. A rise in BNP levels in post-infarct patients during exercise correlates with a rise in left ventricular filling pressure. (Marumoto et. al., 1995)

U.S. Patent No. 6,162,902 describes reagents and methods for the rapid and direct quantification of BNP levels in biological samples. U.S. Pat. No. 6,376,207 describes immunoassays, reagents and methods useful for the rapid and sensitive quantification of the peptide hormone BNP in a biological fluid such as plasma or serum. The Triage® BNP Test, produced by BioSite (San Diego, Calif.) measures BNP levels, using a measure of BNP of over 100-200 pg/mL to diagnose congestive heart failure.

Atherosclerosis (or arteriosclerosis) is the term used to describe progressive luminal narrowing and hardening of the arteries. This disease process can occur in any systemic artery in the human body. For example, atherosclerosis in the arteries that supply the brain can result in stroke. Gangrene may occur when the peripheral arteries are blocked, and coronary artery disease occurs when the arteries that supply oxygen and nutrients to the myocardium are affected.

Coronary artery disease is a multifactorial disease that results in the deposition of atheromatous plaque and progressive luminal narrowing of the arteries that supply the heart muscle. This plaque consists of a mixture of inflammatory and immune cells, fibrous tissue, and fatty material such as low-density lipids (LDL) and modifications thereof, and alpha-lipoprotein. The luminal narrowing or blockage results in reduced ability to deliver oxygen and nutrients to the heart muscle, producing myocardial infarction, angina, unstable angina, and sudden ischemic death as heart failure. Though occlusion usually progresses slowly, blood supply may be cut off suddenly when a portion of the built-up arterial plaque breaks off and lodges somewhere in an artery to block it temporarily, or more usually, when thrombosis occurs within the arterial lumen. Depending on the volume of muscle distal to the blockage during such an attack, a portion of myocardial tissue may die, weakening the heart muscle and often leading to the death of the individual.

Though recent improvements in cardiovascular care have improved the life expectancy of coronary artery disease patients, this has been primarily from improvements in lowering lipid levels, limitation of damage after it has occurred, surgical restoration of blood supply, the suppression of abnormal heart rhythms and prevention of re-infarction. Little improvement has occurred, however, in early prevention of the disease by early diagnosis.

A key problem in treating coronary artery disease is proper diagnosis. Often the first sign of the disease is sudden death due to myocardial ischemia or myocardial infarction. Approximately half of all individuals who die of coronary artery disease die suddenly. Furthermore, for 40-60% of the patients who are eventually diagnosed as having coronary artery disease, myocardial infarction is the first presentation of disease. Unfortunately, approximately 40% of those initial events go unnoticed by the patient. For various reasons, the perception of symptoms by the patient does not correlate well with the total burden of coronary artery disease (Anderson et. al., 1992).

While the causes of atherosclerosis remain unknown, the proper diagnosis of susceptibility may provide patients sufficient time to reduce their risk of developing coronary artery disease. One method to reduce the risk of coronary artery disease is through alteration of patient lifestyle such as smoking cessation, exercise, weight loss, and stress reduction. Other methods include pharmaceutical intervention to treat hypertension, hypercholesterolemia, and diabetes, as well as the use of aspirin. Finally, genetic therapy promises to treat those rare genetic traits that result in a family history of cardiovascular disease (e.g., altered apolipoprotein metabolism).

The ability to identify high-risk individuals would allow physicians to focus preventive measures on those individuals who may gain the greatest benefit, and would provide strong incentives for those at risk to comply with such approaches.

U.S. Pat. No. 5,756,067 notes that tests currently available to measure the risk of developing atherosclerosis include measuring the plasma content of cholesterol, triglycerides, and lipoproteins, but that it is clear that these tests are not conclusive because approximately one-half of heart disease due to atherosclerosis occurs in patients with plasma triglycerides and cholesterol within the normal ranges of the population and because angiographic evidence of atherosclerosis has been found in patients with normal lipid levels.

Exercise stress testing (“EST”) is one of the most commonly used tests in the diagnosis of cardiac conditions such as coronary artery disease (“CAD”). Approximately 5 million people in the United States suffer from CAD, resulting in over 1.5 million heart attacks annually, of which 550,000 are fatal. CAD may be diagnosed when the coronary circulation is insufficient to supply the oxygen and nutrient requirements of the heart muscle, resulting in ischemia. Often a cardiac patient has no symptoms at rest and only develops cardiac symptoms under conditions of cardiac stress. Although changes in the electrocardiogram during EST can be helpful in diagnosis of CAD, the interpretation of the result is limited as they are affected by several conditions, including but not limited to hypertension, left ventricular hypertrophy, cardiac rhythm disorders, medications and gender.

Myocardial perfusion imaging involving the use of radioisotopes is often used in conjunction with EST in order to assist proper diagnosis of CAD. However, such imaging methods are costly, due to the high cost of the radioisotopes and the requirement of personnel with proper training in radioisotope handling. Additionally, in women, breast artifacts can result in inaccurate results. The shifting of breast tissue may lead to the mistaken conclusion that redistribution has occurred.

Stress echocardiography involving direct visualization of the heart function by ultrasound imaging during the time of stress is also used in conjunction with EST in order to assist proper diagnosis of CAD. This diagnostic modality also requires personnel with training and expertise in performing and interpreting echocardiography. Occasionally, it is difficult to obtain adequate views of the heart due to physical factors including obesity and thick chest wall rendering optimum interpretation of the echocardiography result difficult or even impossible. The result is also affected by various underlying cardiac conditions such as valvular heart disease and hypertensive heart disease.

There are various methods other than EST which are used to induce cardiac stress and detect ischemia. These involves administration of pharmacological agents including but not limited to dobutamine, dipyridamole, adenosine and others.

Methods for early diagnosis of CAD before myocardial infarction, which could augment or replace existing methods, are needed.

### BRIEF SUMMARY OF THE INVENTION

An embodiment of the invention is a method for detecting coronary artery disease in a mammal comprising the steps of measuring a baseline level of a marker related to BNP in said mammal; inducing a cardiac stress in said mammal; measuring the marker related to BNP level immediately post cardiac stress; and calculating a relative change in the marker related to BNP level; wherein coronary artery disease is detected in said mammal if the relative change in marker related to BNP level after cardiac stress is greater than a predetermined clinically effective threshold value. In a specific embodiment, the method further comprises the step of measuring the marker related to BNP level about 10-15 minutes post cardiac stress.

In a specific embodiment, the marker related to BNP is BNP, NT pro-BNP, or pre pro BNP.

In another specific embodiment, the measuring of the BNP level comprises an immunoassay.

In one specific embodiment, the mammal is a human. In a further specific embodiment, the human has no known history of a previous myocardial infarction.

In another specific embodiment, the human possesses at least one cardiac risk factor selected from the group consisting of age greater than 35 years, history of smoking, diabetes mellitus, obesity, high blood pressure, high cholesterol, elevated low density lipoproteins and family history of cardiac disease.

In one embodiment of the invention, cardiac stress comprises exercise stress testing. In a specific embodiment, the exercise stress testing comprises a treadmill test. The exercise stress testing comprises a bicycle test in another embodiment of the invention.

In one embodiment of the invention, the method comprises the administration of a myocardial perfusion imaging test, stress echocardiography test, or single-photon emission computed tomography test to the human during cardiac stress.

In one embodiment of the invention, the cardiac stress comprises pharmacologic stress. In a specific embodiment, the pharmacologic stress is induced by the administration of adenosine to the mammal. In another specific embodiment, the pharmacologic stress is induced by the administration of dobutamine to the mammal.

In one embodiment of the invention, the relative change of the marker related to BNP is at least about 10%, or from about 10%-400%. In another embodiment of the invention, the relative change in the marker related to BNP is at least about 1% or at least about 5% per minute of exercise. In another embodiment of the invention, the relative change in the marker related to BNP is from about 5% to about 27% per minute of exercise.

An embodiment of the invention is a method for risk stratification in coronary artery disease in a mammal comprising the steps of measuring a baseline marker related to BNP level in said mammal; inducing a cardiac stress in said mammal; measuring the marker related to BNP level immediately post cardiac stress; and calculating a relative change in the marker related to BNP level; wherein the relative change in the marker related to BNP level correlates with severity of the coronary artery disease, and wherein the higher the relative change, the greater the severity of coronary artery disease.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 shows changes in BNP from baseline to Immediate Post-exercise in patients with and without ischemia; and

FIG. 2 shows receiver operating characteristics of Percent Increase in BNP from baseline to predict presence of Reversible Ischemia on SPECT.

### DETAILED DESCRIPTION OF THE INVENTION

As used herein, the use of the word “a” or “an” when used in conjunction with the term “comprising” in the sentences and/or the specification may mean “one,” but it is also consistent with the meaning of “one or more,” “at least one,” and “one or more than one.” As used herein “another” may mean at least a second or more. Still further, the terms “having”, “including”, “containing” and “comprising” are interchangeable and one of skill in the art is cognizant that these terms are open ended terms.

The term “B type natriuretic peptide” or “BNP” as used herein refers to the mature 32-amino acid B type natriuretic peptide molecule. As described herein, levels of BNP in patient samples can provide an important prognostic indication of future morbidity and mortality. As the skilled artisan will recognize, however, other markers related to BNP may also serve as prognostic indicators in such patients. Examples of “markers related to BNP” are BNP, pre pro BNP, and NT pro BNP. BNP is synthesized as a 108-amino acid pre pro-BNP molecule that is proteolytically processed into a 76-amino acid “NT pro BNP” and the 32-amino acid BNP molecule. Because of its relationship to BNP, the concentration of NT pro-BNP molecule can also provide prognostic information in patients. See, e.g., Fischer et al., Clin. Chem. 47: 591-594 (200 1); Berger et al., J Heart Lung Transplant. 20: 251-(2001).

The phrase “BNP level” or “level of BNP” refers to the amount of BNP measured in a patient sample. In a preferred embodiment, a BNP polypeptide level is measured. Generally, BNP polypeptide levels are expressed as either pg/mL or pmol/L.

As used herein, the term “cardiovascular disease or disorder” refers to disease and disorders related to the cardiovascular or circulatory system. Cardiovascular disease and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skill in the art recognizes that cardiovascular diseases and/or disorders can result from congenital defects, genetic defects, environmental influences (i.e., dietary influences, lifestyle, stress, etc.), and other defects or influences.

As used herein, the term “coronary artery disease” (CAD) refers to a type of cardiovascular disease. CAD is caused by gradual blockage of the coronary arteries. One of skill in the art realizes that in coronary artery disease, atherosclerosis (commonly referred to as “hardening of the arteries”) causes thick patches of fatty tissue to form on the inside of the walls of the coronary arteries. These patches are called plaque. As the plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack.

The term “correlating,” as used herein in reference to the use of prognostic indicators to determine a prognosis, refers to comparing the presence or amount of the prognostic indicator in a patient to its presence or amount in (i) persons known to suffer from, or known to be at risk of, a given condition; (ii) in persons known to be free of a given condition; or (iii) both. For example, a BNP level in a patient can be compared to a level known to be associated with an increased predisposition for an MI or death. The patient's BNP level is said to have been correlated with a prognosis; that is, the skilled artisan can use the patient's BNP level to determine the likelihood that the patient is at risk for an MI or death, and respond accordingly. Alternatively, the patient's BNP level can be compared to a BNP level known to be associated with a good outcome (e.g., no MI, no death, etc.), and determine if the patient's prognosis is predisposed to the good outcome.

As used herein, the term “damaged myocardium” refers to myocardial cells which have been exposed to ischemic conditions. These ischemic conditions may be caused by a myocardial infarction, or other cardiovascular disease or related complaint. The lack of oxygen causes the death of the cells in the surrounding area, leaving an infarct, which will eventually scar.

As used herein, the term “heart failure” refers to the pathophysiological state in which the heart is unable to pump blood at a rate commensurate with the requirements of the metabolizing tissues or can do so only from an elevated filling pressure.

As used herein, the phrase “immediately after termination of cardiac stress” or “immediately after termination of stress” refers to a time period within 3 minutes of the termination of cardiac stress. As used herein, the phrase “immediately after termination of pharmacologic stress” refers to a time period within 3 minutes of the termination of pharmacologic stress. One with skill in the art realizes that the cessation of pharmacologic stress may be due to insufficient concentrations pharmacologic stress-causing agent in the circulation due to metabolism of said agent, or it may be caused by administration of a reversing agent. As used herein, the phrase “immediately after termination of exercise” refers to a time period within 3 minutes of the termination of exercise. One with skill in the art also realizes that samples can be taken from patients at specific time points, and then frozen for later analysis, for example at −80° C.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “major adverse coronary events” or “MACE” refers in-hospital mortality, Q-wave myocardial infarction, emergency coronary artery bypass surgery (CABG) within 24 hours of receiving Percutaneous Coronary Intervention, hospital admission for revascularization, and refractory angina pectoris.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Mammals may be referred to as “patients”.

The phrase “marker related to BNP” refers to any polypeptide that originates from the pre pro-BNP molecule, including the 32-amino acid BNP molecule. Thus, a marker related to or associated with BNP includes BNP, the NT pro-BNP molecule, the pro domain, a fragment of BNP that is smaller than the entire 32-amino acid sequence, a fragment of pre pro-BNP other than BNP, and a fragment of the pro domain. One skilled in the art will also recognize that the circulation contains proteases which can proteolyze BNP and BNP related molecules and that these proteolyzed molecules (peptides) are also considered to be “BNP related” and are additionally subjects of this invention. Additionally, a marker related to BNP can be an mRNA transcript of pre-pro-BNP.

The term “patient sample” refers to a sample obtained from a living mammal for the purpose of diagnosis, prognosis, or evaluation. In certain embodiments, such a sample may be obtained for the purpose of determining the outcome of an ongoing condition or the effect of a treatment regimen on a condition. Preferred patient samples are blood samples, serum samples, plasma samples, cerebrospinal fluid, and urine samples.

“Predictive value” is the probability that a mammal with a positive test has the disease and one with a negative test result does not have the disease. It may be determined by the prevalence of the condition and the sensitivity and specificity of the test. It can be expressed in two ways, as either the positive predictive value or the negative predictive value. Prevalence is a measure of the incidence of disease in a population at one given time, e.g. cases per 100,000. In certain embodiments, one or more additional prognostic indicators can be combined with a level of BNP, or a related marker, in a patient sample to increase the predictive value of BNP or the related marker as a prognostic indicator. The phrase “increases the predictive value” refers to the ability of two or more combined prognostic indicators to improve the ability to predict a given outcome, in comparison to a prediction obtained from any of the prognostic indicators alone.

The “relative change in BNP level” is the change in the BNP level immediately after exercise as compared to the baseline level. In one embodiment, a relative BNP level change is expressed as the ratio of the difference between peak BNP and baseline BNP to baseline BNP i.e. (peak BNP-baseline BNP)/(baseline BNP). In another embodiment, the relative BNP level change is expressed as a percentage change of the baseline BNP compared to BNP immediately after exercise. In another embodiment, the relative BNP level change is expressed as a percentage change of the baseline BNP compared to BNP immediately after exercise, per minute of cardiac stress. A “baseline BNP” level is the BNP level before a specific event. For example, the BNP level after exercise is compared to a baseline BNP level before exercise.

As used herein, the term “risk factors” refers to factors which increase the likelihood of developing coronary artery disease. These risk factors include, but are not limited to, age greater than 35 years, history of smoking, diabetes mellitus, obesity, high blood pressure, high cholesterol, elevated low density lipoproteins and family history of cardiac disease.

The term “risk stratification” as used herein refers to identifying individuals at particularly high risk for a clinical outcome. In some embodiments, risk stratification helps to determine the severity of coronary artery disease in an individual. As used herein, “severity of coronary artery disease” refers to the likelihood of negative clinical outcomes. Severity of coronary artery disease also refers to the location and size of coronary artery blockages. In certain embodiments, risk stratification in individuals diagnosed with coronary artery disease before myocardial infarction helps to predict the likelihood of a future myocardial infarction. In a preferred embodiment, risk stratification for CAD correlates to the relative change in BNP. Examples of risk stratification systems are the Euroscore system and the Parsonnet sytem, which assess risk during cardiac surgery.

1. Clinically Effective Threshold Value of the Relative Change in BNP

The sensitivity of a diagnostic test is a measure of the proportion of mammals with coronary artery disease having a positive test result. The specificity of a diagnostic test is a measure of proportion of mammals without coronary artery disease having a negative test result. One with skill in the art realizes that in can be desirable to lower or raise the sensitivity or specificity of a given test within a clinically acceptable range, depending on the purpose of the test. The sensitivity and specificity may be arbitrarily set to a certain value, depending on the purpose of the test. In certain embodiments of the invention, the sensitivity of the invention(relative change in BNP during cardiac stress) in detecting reversible myocardial ischemia as evidenced by positive myocardial perfusion scan or stress echocardiography is within the range of 10%-90% depending on the cut-off levels of relative change in BNP.

Operating characteristics of diagnostic tests and procedures are measures of the technical performance of these technologies. A means of expressing these values of the diagnostic test of the present invention is with a receiver operating characteristic (ROC) curve, which plots the relationship between the true positive ratio, the sensitivity, and false positive ratio (1-specificity) as a function of the cut-off level of a disease marker. ROC curves help to demonstrate how raising or lowering the cut-off point for defining a positive test result affects tradeoffs between correctly identifying people with a disease (true positives) and incorrectly labeling a person as positive who does not have the condition (false positives).

Taken alone, sensitivity and specificity do not reveal the probability that a given patient really has a disease if the test is positive, or the probability that a given patient does not have the disease if the test is negative. These probabilities are captured by two other operating characteristics. Predictive value positive is the proportion of those patients with a positive test result who actually have the disease. Predictive value negative is the proportion of patients with a negative test result who actually do not have the disease. Unlike sensitivity and specificity, predictive value positive and predictive value negative are not constant performance characteristics of a diagnostic test; they change with the prevalence of the disease in the population of interest. For example, if the disease is sufficiently rare in the population, even tests with high sensitivity and high specificity can have low predictive value positive, generating more false-positive than false negative results.

In accordance with standard practice, BNP levels are measured against minimum clinically effective threshold values. The “diagnostic accuracy” or “clinical efficacy” of a test, assay, or method concerns the ability of the test, assay, or method to distinguish between patients having a disease, condition, or syndrome and patients not having that disease, condition, or syndrome based on whether the patients have a “clinically significant presence” of relative BNP changes. By “clinically significant presence” is meant that the change of the relative BNP levels in the patient sample is higher than the predetermined cut-off point, or “threshold value”, relative BNP changes and therefore indicates that the patient has the disease, condition, or syndrome for which the sufficiently high presence of that relative BNP changes is a marker.

The term “clinically effective threshold value” refers to relative changes in BNP levels with a predetermined threshold value correctly indicating the presence or absence of the disease, condition, or syndrome. Changing the cut-off point, or threshold value of a test, changes the sensitivity and specificity in a qualitatively inverse relationship. For example, if the threshold value is lowered, more individuals in the population tested will typically have test results over the cutoff point or threshold value. Accordingly, the sensitivity of the test will be increased. However, at the same time, there will be more false positives because more people who do not have the disease, condition, or syndrome will be indicated by the test to have relative BNP changes above the threshold value and therefore to be reported as positive rather than being correctly indicated by the test to be negative. Accordingly, the specificity of the test will be decreased. Similarly, raising the cut-off point will tend to decrease the sensitivity and increase the specificity. Therefore, in assessing the accuracy and usefulness of a proposed medical test, assay, or method for assessing a patient's condition, one should always take both sensitivity and specificity into account and be mindful of what the threshold value is at which the sensitivity and specificity are being reported because sensitivity and specificity may vary significantly over the range of threshold values. One with skill in the art realizes that in certain situations, such as screening, it is desirable to increase the sensitivity of the test by lowering the threshold value. In other embodiments, it may be desirable to increase the specificity and decrease the sensitivity of the test by increasing the threshold value. In one embodiment of the present invention, the degree of relative change in BNP levels after cardiac stress serves as a prognostic indicator of the severity of underlying coronary artery disease. In one embodiment of the invention, the invention provides a test for CAD that has a sensitivity of at least 10%, 20%, 30%, 40%, 60%, 70%, 80%, or 90%. In an embodiment of the invention, the invention provides a test for CAD that has a sensitivity of at least 70% and a specificity of at least 50%. One with skill in the art realizes that in certain embodiments of the invention, it is appropriate to have any threshold value that provides either a sensitivity or a specificity of up to 100%.

2. Cardiac Stress

Stress tests are non-invasive procedures whereby the heart muscle is exercised so that the electrical activity of the heart is monitored under conditions of physical stress. The cardiac changes elicited by stress include increased heart rate, increased cardiac output, increased stroke volume due to increased venous return and increased myocardial contractility, and rise in systolic blood pressure. These changes increase the heart's need for oxygen, and therefore increase the need for coronary blood flow, creating a diagnostically revealing response for detection of CAD.

In one embodiment of the invention, cardiac stress can unmask coronary artery disease in otherwise asymptomatic patients. Stress-induced reversible ischemia occurs in patients who at rest have normal blood flow to the heart muscle, but during peak exercise, have reduced blood flow to the heart. The present invention describes a noninvasive test for BNP levels after cardiac stress, the results of which significantly correlate with the presence of reversible ischemia as determined by myocardial perfusion imaging.

The heart may be stressed by having a patient exercise on a treadmill or a stationary bicycle. If the patient is unable to exercise secondary to physical limitations such as severe arthritis, artificial limbs, generalized weakness, paralysis, unsteady gait, etc., the physician may choose a pharmacological or chemical form of test. In this case, a compound is given intravenously to perform a nearly comparable degree of cardiac stress. If possible, some form of pharmacological stress testing may be combined with a brief period of treadmill exercise. Patients may be exercised using the standard Bruce protocol.

The Bruce protocol is the most common protocol used in the United States. This protocol specifies the speed and level of the incline of a motor driven treadmill during a total of seven three-minute exercise states with no rest periods. The test is defined as stopped when any of the following occur: when the protocol is completed; when the patient reaches a pre-set heart rate goal; when the patient experiences acute discomfort; when a diagnostic change occurs in the EGG or blood pressure; or when the patient fatigues.

In certain embodiments of the invention, BNP levels are measured (i) immediately before exercise while at rest, (ii) “immediately” after termination of exercise, and (iii) after 10-15 minutes of rest after termination of exercise. As defined herein, termination of exercise stress occurs when the test is stopped, as described above.

For patients with exclusions to treadmill testing or for those who cannot perform at least 85% of predicted maximal exercise or normal treadmill times, pharmacologic stress testing may be performed using either adenosine or dobutamine, or any other appropriate cardiac stress-inducing pharmacalogic agent such as dipyridamole. BNP levels can be measured (i) immediately before pharmacologic stress while at rest, (ii) “immediately” after termination of pharmacologic stress, and (iii) after 10-15 minutes of rest after termination of pharmacologic stress. Pharmacologic stress is defined as terminated, or stopped, when a reversing agent is administered, or when the amount of circulating active drug in the patient is no longer sufficient to cause cardiac stress.

Adenosine is a potent coronary vasodilator and is currently a preferred agent for pharmacologic stress testing since it has a very reproducible hemodynamic and pharmacologic profile. Dobutamine is used if a patient has a contraindication to adenosine. In certain embodiments of the invention, a pharmacologic stress test is considered stopped when the pharmacologic agent no longer is creating cardiac stress, either because there is no longer sufficient concentration of active drug in the system to cause stress, or because another agent has been administered to reverse the stress, such as aminophyllin.

In certain embodiments of the invention, dipyridamole is infused at a rate of 0.142 mg/kg/minute for 4 minutes through a large vein. A myocardial perfusion agent is injected 2 to 4 minutes following completion of the infusion (typically at 7 minutes) or sooner if impressive hemodynamic side effects are noted.

In certain embodiments of the invention, adenosine is infused at an infusion rate of 140 ug/kg/minute for a 6 minute infusion. Maximal vasodilatation is generally observed within 2 minutes of initiation of the infusion. The infusion rate may be reduced to 75-100 ug/kg if the patient experiences severe side effects and the response is almost instantaneous. Early termination of the infusion should be considered for patients that develop severe hypotension (BP systolic less than 90 mm Hg), wheezing, chest pain associated with ECG evidence of ischemia (ST depression over 2 mm), and in patients that develop persistent second degree or complete heart block. Aminophyllin is not necessary to reverse the adenosine due to the extremely short half-life of adenosine (2-10 sec.).

In certain embodiments, dobutamine is given intravenously by a graded infusion beginning at 10 ug/kg/min. and increased by 10 ug every 3 minutes to a maximum infusion of 40 ug/kg/min. The infusion should be terminated if the patient develops a ventricular tachycardia or ST segment elevation. Atropine can be used to augment increasing the heart rate. Atropine is a parasympatholytic agent that blocks the cardiac action of the vagus nerve and it augments myocardial oxygen consumption by increasing the heart rate. The onset of action peaks in 2 to 3 minutes. Atropine 0.6 mg is injected intravenously and can be administered in incremental doses up to a maximum of 2 mg. A beta blocker such as metoprolol 5 mg intravenously reverses the effects of atropine and can also be used to reverse the effects of dobutamine.

In certain embodiments, myocardial perfusion imaging may be performed with cardiac stress testing and testing for relative BNP changes. For this test, patients undergo stress testing (either treadmill or pharmacologic) and receive a radioisotope, such as thallium-201, at peak exercise. In dual isotope testing, patients receive an injection of a radioisotope, such as thallium-201, at rest followed by rest imaging within 10-15 minutes. Immediately after the rest imaging they undergo stress (either treadmill or pharmacologic) and receive a different radioisotope, such as technetium-99m sestamibi (Cardiolite®), at peak stress. They are then re-imaged 30 to 60 minutes later. BNP levels are measured before, immediately after, and 10-15 minutes after cardiac stress.

In another embodiment of the invention, stress echocardiography may be used in conjunction with testing for changes in relative BNP levels. Stress echocardiography is used to detect reversible myocardial ischemia by detecting regional abnormalities in wall motion during or immediately following exercise or during pharmacological stressing using dobutamine. However, in some pre-infarct patients with single valve blockage, stress echocardiography is inconclusive, and one embodiment of the invention augments this testing with BNP level measurements before and after exercise to aid diagnosis of coronary artery disease in patients without known myocardial infarctions.

In one embodiment of the invention, SPECT, or Single Photon Emission Tomography perfusion scan, imaging is performed together with stress testing. SPECT imaging assesses blood flow in the heart and is performed. A radioactive tracer is injected into the blood and a series of images are captured. Computer software creates a picture of the heart by assembling the images.

3. Immunodetection Methods for BNP

In still further embodiments, the present invention concerns immunodetection methods for binding, purifying, removing, quantifying and/or otherwise generally detecting biological components such as BNP or markers related to BNP as expressed message(s), protein(s), polypeptide(s) or peptide(s). U.S. Pat. No. 6,162,902 described an immunoassay for human BNP as well as antibody compositions for antibodies specific to BNP. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev O, 1999; Gulbis B and Galand P, 1993; and De Jager R et al., 1993, each incorporated herein by reference.

In general, the immunobinding methods include obtaining a patient sample suspected of containing BNP protein, polypeptide and/or peptide, and contacting the sample with a first anti-BNP message and/or anti-BNP translated product antibody in accordance with the present invention, as the case may be, under conditions effective to allow the formation of immunocomplexes.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the BNP produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any BNP antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The BNP antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

3. Kits for the Detection of BNP

BNP levels may be measured through the use of a kit. The kits may comprise a suitably aliquoted anti-BNP antibody, whether labeled or unlabeled, as may be used to prepare a standard curve for a detection assay. An example of an appropriate kit for measuring BNP is the Triage®) BNP Test, produced by BioSite (San Diego, Calif.). The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the anti-BNP antibody, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the anti-BNP antibody formulations are placed, preferably, suitably allocated. The kits may also comprise a second container means for containing a sterile, pharmaceutically acceptable buffer and/or other diluent.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Study Design

Patients who were undergoing Exercise treadmill myocardial perfusion scan were enrolled. BNP samples were drawn at (i) before exercise while at rest (ii) immediately after exercise termination, and (iii) 10-15 min post exercise. The BioSite Triage kit was used to measure BNP. The measurable range of the BioSite kit is 5 pg/mL to 1300 pg/mL. A relative BNP level was assigned to each patient based on difference from baseline BNP after exercise.

Sixty consecutive patients undergoing myocardial perfusion tomography (SPECT) in conjunction with Bruce protocol treadmill exercise for evaluation of chest pain or screening for ischemia were enrolled into our study. Exclusion criteria were defined as: clinical heart failure (NYHA class II and above), known left ventricular ejection fraction <40%, unstable angina, recent evidence of myocardial infarction (less than 6 weeks), and known moderate to severe aortic or mitral valve disease.

### Example 2

### BNP Measurements

Before beginning of treadmill exercise, a 21 Gauge intravenous catheter was placed in a forearm vein and blood samples were drawn at rest, immediate post exercise and 10-15 minutes post exercise. The catheter was flushed with at least 5 cc of normal saline prior to collection and the first 5 cc of blood drawn were discarded prior to collection. The venous blood was collected in an EDTA tube and analyzed within 4 hours of collection. If the testing could not be completed within 4 hours, the plasma was separated and stored at −70 degrees C. until it was tested. B-type natriuretic peptide was measured with use of a fluorescence immunoassay kit (Triage, Biosite) for the quantitative determination of BNP in whole-blood and plasma specimens. The lowest detectable measurement for this assay was 5 pg/mL. The inter-assay coefficient of variation was 10.1% for 28.8 pg/mL, 12.4% for 586 pg/mL and 16.2% for 1180 pg/mL.

Example 3

### Exercise SPECT Studies

Myocardial perfusion was performed by quantitative Tc-99m Sestamibi myocardial single-photon emission computed tomography (SPECT) and post-stress left ventricular ejection fraction was performed by standard gated SPECT acquisition protocol using either single- or double-headed detector systems (see Grines et. al, 2003) in a blinded manner.

### Example 4

### Statistical Analysis

Parametric variables were compared with Student t test and non-parametric variables were compared with rank sum test. Categorical variables were compared with Chi square test. Fisher exact test was used when observed frequency was less than 5. Friedman repeated measures analysis of variance on ranks was used to analyze the effect of exercise on BNP level at rest, immediately post, and 10-15 minutes post exercise. If statistical significance was found, post hoc Tukey test was performed.

Stepwise regression analysis was used to determine the independent effect of a range of factors on the presence of reversible ischemia in SPECT and the change in BNP. The factors included were age, sex, exercise duration, maximum METS achieved, peak heart rate, peak systolic and diastolic blood pressure, BNP level at rest, BNP level at immediate post exercise, BNP level at 10-15 minutes post exercise, absolute change in BNP from rest to immediate post exercise, percent change in BNP from to immediate post exercise, percent change in BNP per minute of exercise, ST segment changes and peak ejection fraction. The predictive power of the percent change in BNP per minute of exercise for detection of ischemia by SPECT was evaluated using receiver operating characteristic (ROC). A p value of <0.05 was defined as significant. Analyses were performed with SigmaStat (version 2.03) software and ROC analysis was performed using MedCalc.

### Example 5

### Patient Population

Sixty consecutive patients who met the criteria for inclusion were enrolled in the study. There were 41 men and 19 women, with a mean age of 57.2±9.3 years. Diabetes was present in 18%. Twenty three percent were on betablockers, 15% on angiotensin converting enzyme inhibitors, and 11% on calcium channel blockers.

Ten patients, all men, had reversible myocardial perfusion defects by SPECT (mean defect size 14%, range 5-37%). Post-exercise ejection fraction averaged 50.6±8%. Table 1 depicts the exercise stress test parameters in patients with and without ischemia. These parameters were not different between subjects with and without evidence of ischemia.

TABLE 1

| | Reversible Ischemia per SPECT | | |
|---|---|---|---|
| | during Treadmill Exercise | | |
| | No ischemia | Ischemia | P |
| Age, years | 56.9 ± 9.5 | 58.7 ± 8.2 | 0.59 |
| Male Sex, % | 62 | 100 | 0.02 |
| Hypertension, % | 38 | 50 | 0.67 |
| Diabetes, % | 18 | 20 | 1.00 |
| Current smoking, % | 4 | 0 | 1 |
| Previous myocardial infarction, % | 4 | 20 | 0.126 |
| Previous revascularization, % | 14 | 40 | 0.101 |
| Current beta-blocker usage, % | 22 | 30 | 0.68 |
| Current ACEI, % | 12 | 30 | 0.16 |
| Current calcium antagonist use, % | 10 | 20 | 0.33 |
| Exercise time, minutes | 8.4 | 8.5 | 0.86 |
| Peak systolic BP, mmHg | 169 | 179 | 0.3 |
| Peak diastolic BP, mmHg | 79 | 86 | 0.19 |
| Peak heart rate/min | 156 | 150 | 0.48 |
| ST depression on ECG, % | 20 | 40 | 0.22 |
| BNP at baseline, pg/mL (median, | 15.05 | 13.4 | 0.797 |
| 25^{th}, 75^{th}) | (7, 37.7) | (9.5, 30.6) | |
| BNP immediate post exercise, | 34.7 | 26.6 | 0.579 |
| pg/mL (median, 25^{th}, 75^{th}) | (14.9, 67.6) | (9.5, 30.6) | |
| BNP 10-15 minutes post exercise, | 20.3 | 15.6 | 0.96 |
| pg/mL (median, 25^{th}, 75^{th}) | (8.6, 48.5) | (13, 37.4) | |
| Absolute rise in BNP pg/mL, | 10.4 | 15.5 | 0.115 |
| median, 25^{th} and 75^{th} | (3.6, 10) | (10-36.5) | |
| Percent increase in BNP from | 67 | 112.5 | 0.02* |
| baseline, %, (median, 25^{th}, 75^{th}) | (26, 101) | (86, 146) | |
| Percent increase in BNP per | 7 ± 0.12 | 14 ± .23 | 0.014* |
| minute of exercise adjusted for | | | |
| gender, %, (mean ± SD) | | | |
| Peak ejection fraction, % (mean ± | 64.1 ± 1.5 | 50.7 ± 2.5 | <0.001 |
| SEM) | | | |

### Example 6

### Changes in BNP During Exercise in Individuals without Ischemia

In subjects without evidence of ischemia, BNP levels increased from a baseline median level of 15.05 pg/mL (interquartile range, 7 to 37.7) to a median of 34.7 pg/mL (interquartile range, 14.9 to 67.6) immediately post-exercise. At 10-15 minutes post exercise, BNP levels decreased towards baseline values (median 20.3 pg/mL; interquartile range 8.6 to 48.5). This pattern of increase and decrease in the level of BNP was significant (p<0.001). This pattern of significant rise and fall in BNP levels immediately post and later after exercise was observed in both normal men and women (p<0.001). Although BNP levels throughout the exercise protocol tended to be higher in women compared to men, the difference between gender did not reach statistical significance [BNP women vs. men: Baseline 20 (12.5-60.1) vs. 12.5 (5.9-30.8); immediately post exercise: 38.5 (18.2-72.6) vs. 22.3 (9.25-53.3); 10-15 min post: 26 (14.1-78.25) vs. 17.4 (8.15-42.5)].

### Example 7

### Changes in BNP During Exercise in Individuals with Ischemia

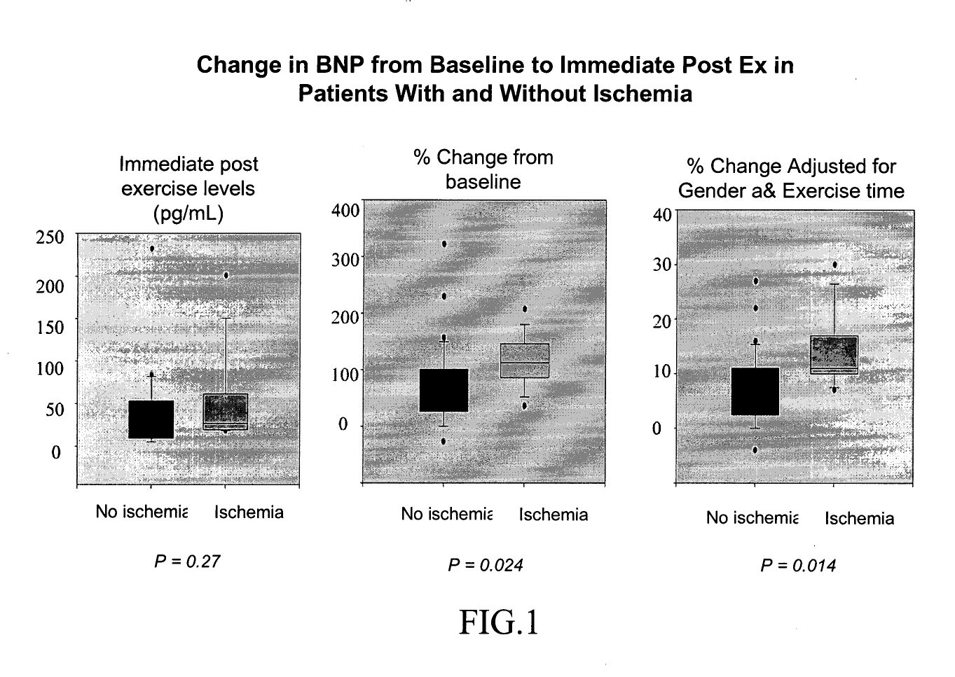

In subjects with evidence of ischemia, BNP levels also increased from baseline to immediately post-exercise [13.4 pg/mL (9.5-30.6) vs. 26.7 (19.3-61.5)] and decreased towards baseline [15.6 pg/mL (13-37.4)] within 10-15 minutes (p<0.001). Neither absolute BNP levels at peak nor the absolute level of rise from baseline to immediate post-exercise differentiated between ischemic and non-ischemic patients (p=0.27 and 0.115). However, if baseline values were accounted for and if the data was depicted as percent change from baseline, the difference between the two groups was significant (p=0.024) (FIG. 1). The best distinction between ischemic and non-ischemic patients was observed when percent change in BNP was adjusted for gender and exercise time. The mean rise in BNP in the non-ischemic patients was 7.7%/min of exercise (SEM±1%), while in the ischemic patients it was 14%/min (SEM±2%) (p=0.014; FIG. 1).

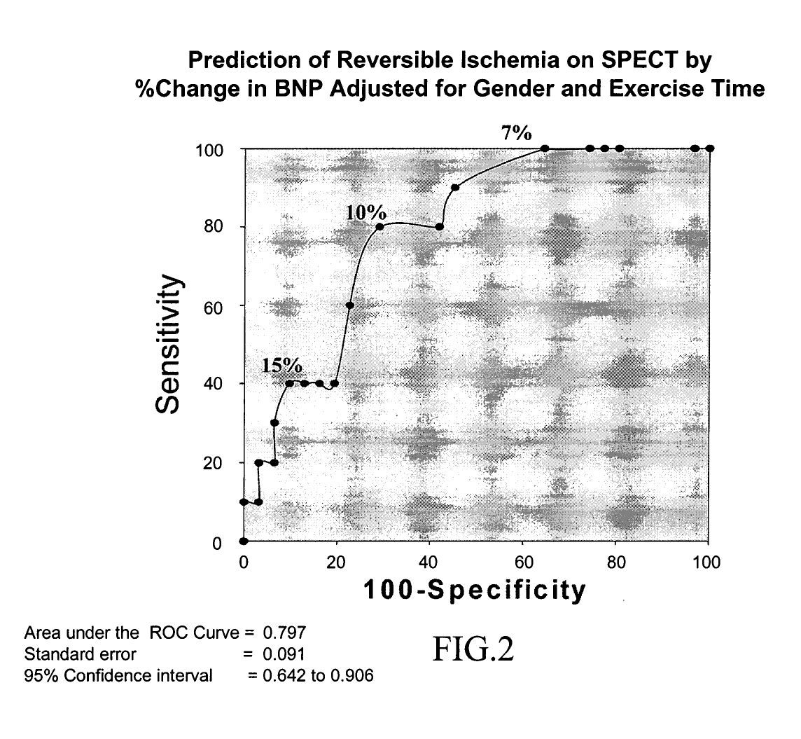

Multivariate analysis showed that percent change in BNP per minute of exercise (p=0.021) and post exercise ejection fraction (p=0.024) were independent predictors of reversible ischemia per SPECT in men. Level of BNP at rest, immediately post exercise or 10-15 minutes post exercise, and the absolute rise in BNP from rest to immediate post-exercise did not significantly add to the prediction of reversible ischemia by SPECT. ROC analysis of the percent change in BNP adjusted for gender and exercise time revealed an area under the curve of 0.797 (SE=0.091) with 95% confidence interval of 0.642 to 0.906. The cut off point of 10% rise in BNP from baseline for each minute of exercise in this analysis had a sensitivity of 80% and a specificity of 71% in detecting reversible ischemia (FIG. 2). See Table 2 and Table 3 for sensitivity and specificity of various cut-off points in BNP rise, shown as both percent rise, and adjusted for time of cardiac stress.

TABLE 2
Percent Rise in BNP

| Percent | | | | | | |
|---|---|---|---|---|---|---|
| rise | Sensitivity | | Specificity | | | |
| BNP | (95% CF) | | (95% CF) | | PPV (%) | NPV (%) |
| 31 | 100 | (69-100) | 28 | (16.2-42.5) | 21 | 100 |
| 36 | 90 | (55.5-98.3) | 28 | (16.2-42.5) | 20 | 93.3 |
| 61 | 90 | (55.5-98.3) | 50 | (35.5-64.5) | 26.5 | 96.2 |
| 84 | 80 | (44.4-96.9) | 62 | (47.2-75.3) | 29.6 | 93.9 |
| 92 | 70 | (34.8-93) | 70 | (57.5-83.8) | 30 | 90 |
| 103 | 60 | (26.4-87.6) | 80 | (66.3-90) | 33.3 | 88.9 |
| 125 | 40 | (12.4-73.6) | 84 | (70.9) | 33.3 | 87.5 |

TABLE 3
Percent Rise in BNP/minute

| Percent | | | | |
|---|---|---|---|---|
| Rise/min | | | | |
| stress | Sens. (95% C.I.) | Spec. (95% C.I.) | PPV | NPV |
| 4 | 100.0 (69.0-100.0) | 0.0 (0.0-11.3) | 24.4 | 100 |
| 6 | 100.0 (69.0-100.0) | 45.2 (27.3-64.0) | 37.0 | 100 |
| 7 | 90.0 (55.5-98.3) | 51.6 (33.1-69.8) | 37.5 | 94.1 |
| 9 | 80.0 (44.4-96.9) | 58.1 (39.1-75.4) | 38.1 | 90.0 |
| 1 | 60.0 (26.4-87.6) | 71.0 (52.0-85.7) | 40.0 | 84.6 |
| 14 | 40.0 (12.4-73.6) | 87.1 (70.1-96.3) | 50.0 | 81.8 |
| 16 | 30.0 (7.0-65.2) | 93.5 (78.5-99.0) | 60.0 | 80.6 |
| 22 | 20.0 (3.1-55.6) | 96.8 (83.2-99.5) | 66.7 | 78.9 |
| 27 | 10.0 (1.7-44.5) | 100.0 (88.7-100.0) | 100 | 77.5 |

Sens. = Sensitivity
Spec. = Specificity
PPV = Positive predictive value
NPV = Negative predictive value
ROC analysis of percent change
POSITIVE GROUP
Sample size = 10
NEGATIVE GROUP
Sample size = 50
Disease prevalence (%) = 16.7
Area under the ROC curve = 0.729
Standard error = 0.097
95% Confidence interval = 0.599 to 0.836

### Example 8

### NT pro BNP Levels in Patients with Reversible Ischemia

The protocol used for measuring relative changes in BNP as described above is also used in testing the value of relative change in NT pro BNP level during cardiac stress in diagnosis of reversible myocardial ischemia.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### References

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Patents:

U.S. Pat. No. 6,162,902

U.S. Pat. No. 6,376,207

U.S. Pat. No. 3,817,837

U.S. Pat. No. 3,850,752

U.S. Pat. No. 3,939,350

U.S. Pat. No. 3,996,345

U.S. Pat. No. 4,277,437

U.S. Pat. No. 4,275,149

U.S. Pat. No. 4,366,241

Non-patent Publications:

Anderson, H. V. et. al, Am Heart J 123, 1312-23 (1992).

Berger, R. et. al, J Heart Lung Transplant. 20, 251 (2001).

Crilley J G, Farrer M. Heart. 86: 638-642 (2001).

De Jager, R. Semin Nucl Med. 23(2),165-79 (1993).

De Lemos, J. A. et. al, N Engl J Med. 345, 1014-21(2001).

Doolittle, M. H. et. al, Methods Mol Biol. 109, 215-371 (1999).

Fischer, Y. et. al, Clin. Chem. 47, 591-594 (2001).

Goetze J P, Christoffersen C, Perko M, Arendrup H, Rehfeld J F, Kastrup J, Nielsen L B. FASEB J. 2003 June;17(9):1105-7. Epub 2003 Apr. 22 (2003).

Grines C L, Watkins M W, Mahmarian J J, Iskandrian A E, Rade J J, Marrott P, Pratt C, Kleiman N J Am Coll Cardiol. October 15;42(8):1339-47 (2003).

Gulbis, B. Hum Pathol. et. al, 24(12),1271-85 (1993).

Huang W S, Lee M S, Perng H W, Yang S P, Kuo S W, Chang H D Nov; 51(11):1423-6 (2002).

Kikuta K, Yasue H, Yoshimura M, Morita E, Sumida H, Kato H, Kugiyama K, Ogawa H, Okumura K, Ogawa Y, Nakao Am Heart J. Jul; 132(1 Pt 1): 101-7 (1996).

Kirsten Bibbins-Domingo, Maria Ansari, Nelson B. Schiller, Barry Massie, and Mary A. Whooley. B-Type Dec; 108: 2987 -2992 (2003).

Maeda K, Tsutamoto T, Wada A, et. al, Am Heart J.; 135: 825-832 (1998).

Maisel A. S., Krishnaswamy P., Nowak R. M., McCord J., Hollander J. E., Duc P., Omland T., Storrow A. B., Abraham W. T., Wu A. H. B., Clopton P., Steg P. G., Westheim A., Knudsen C. W., Perez A., Kazanegra R., Herrmann H. C., McCullough P. A., N Engl J Med 2002; 347:161-167, July 18, (2002).

Marumoto, J. M. Jpn Circ J. 59(11), 715-24 (1995).

Marumoto K, Hamada M, Hiwada K. Clin Sci (Lond); 88: 551-556 (1995).

Nagaya N, Nishikimi T, Okano Y, et al Cardiol. January; 31(1): 202-8 (1998).

Nicholson S, Richards M, Espiner E, Nicholls G, Yandle T July-August; 20(7-8): 535-40 (1993).

Qi W, Mathisen P, Kjekshus J, Simonsen S, et al. Am Heart J. Oct; 142(4): 725-32 (2001).

Omland, T. Circulation. 1;93(11), 1963-9 (1996).

Omland T, de Lemos J A, Morrow D A, et al, Am J Cardiol; 89: 463-465 (2002).

Onuoha G N, Nicholls D P, Patterson A, Beringer T Aug; 32(4): 319-25 (1998).

Sutton T M, Stewart R A, Gerber I L, West T M, Richards A M, Yandle T G, Kerr A J. J Am Coll Cardiol. 2003 Jun. 18; 41(12): 2280-7 (2003).

Stein, B. C. Am Heart J.135(5 Pt 1), 914-23 (1998).

Yoshimura M, Yasue H, Okumura K, et al. Circulation; 87: 464-469 (1993).

What is claimed is:

1. A method for detecting coronary artery disease in a mammal comprising the steps of:
measuring a baseline level of a marker related to BNP in said mammal;
inducing a cardiac stress in said mammal;
measuring the marker related to BNP level immediately post cardiac stress; and
calculating a relative change in the marker related to BNP level; wherein coronary artery disease is detected in said mammal if the relative change in marker related to BNP level after cardiac stress is greater than a predetermined clinically effective threshold value.

2. The method of claim 1, wherein the marker related to BNP is BNP, NT pro-BNP, or pre pro BNP.

3. The method of claim 1, wherein the marker related to BNP is BNP.

4. The method of claim 1, wherein the measuring of the BNP level comprises an immunoassay.

5. The method of claim 1, further comprising the step of measuring the marker related to BNP level about 10-15 minutes post cardiac stress.

6. The method of claim 1, wherein said mammal is a human.

7. The method of claim 6, wherein said human has no known history of a previous myocardial infarction.

8. The method of claim 6, wherein said human possesses at least one cardiac risk factor selected from the group consisting of age greater than 35 years, history of smoking, diabetes mellitus, obesity, high blood pressure, high cholesterol, elevated low density lipoproteins and family history of cardiac disease.

9. The method of claim 1, wherein the cardiac stress comprises exercise stress testing.

10. The method of claim 9, wherein the exercise stress testing comprises a treadmill test.

11. The method of claim 9, wherein the exercise stress testing comprises a bicycle test.

12. The method of claim 1, further comprising the administration of a myocardial perfusion imaging test to a human during said cardiac stress.

13. The method of claim 1, further comprising the administration of a stress echocardiography test to a human during said cardiac stress.

14. The method of claim 1, further comprising the administration of a single-photon emission computed tomography test to a human during said cardiac stress.

15. The method of claim 1, wherein the cardiac stress comprises pharmacologic stress.

16. The method of claim 15, wherein the pharmacologic stress is induced by the administration of adenosine to the mammal.

17. The method of claim 15, wherein the pharmacologic stress is induced by the administration of dobutamine to the mammal.

18. The method of claim 1, wherein the relative change of the marker related to BNP is at least about 10%.

19. The method of claim 1, wherein the relative change of the marker related to BNP is from about 10% to about 400%.

20. The method of claim 1, wherein the relative change in the marker related to BNP is at least about 1% per minute of exercise.

21. The method of claim 20, wherein the relative change in the marker related to BNP is at least about 5% per minute of exercise.

22. The method of claim 20, wherein the relative change in the marker related to BNP is from about 5% to about 27% per minute of exercise.

23. A method for risk stratification in coronary artery disease in a mammal comprising the steps of:
measuring a baseline marker related to BNP level in said mammal;
inducing a cardiac stress in said mammal;
measuring the marker related to BNP level immediately post cardiac stress; and
calculating a relative change in the marker related to BNP level; wherein the relative change in the marker related to BNP level correlates with severity of the coronary artery disease, and wherein the higher the relative change, the greater the severity of coronary artery disease.

24. The method of claim 23, wherein the marker related to BNP is BNP, NT pro-BNP, or pre pro BNP.

25. The method of claim 23, wherein the marker related to BNP is BNP.

26. The method of claim 23, wherein the measuring of the BNP level comprises an immunoassay.

27. The method of claim 23, further comprising the step of measuring the marker related to BNP level about 10-15 minutes post cardiac stress.

28. The method of claim 23, wherein said mammal is a human.

29. The method of claim 28, wherein said human has no known history of a previous myocardial infarction.

30. The method of claim 28, wherein said human possesses at least one cardiac risk factor selected from the group consisting of age greater than 35 years, history of smoking, diabetes mellitus, obesity, high blood pressure, high cholesterol, elevated low density lipoproteins and family history of cardiac disease.

31. The method of claim 23, wherein the cardiac stress comprises exercise stress testing.

32. The method of claim 31, wherein the exercise stress testing comprises a treadmill test.

33. The method of claim 31, wherein the exercise stress testing comprises a bicycle test.

34. The method of claim 23, further comprising the administration of a myocardial perfusion imaging test to a human during said cardiac stress.

35. The method of claim 23, further comprising the administration of a stress echocardiography test to a human during said cardiac stress.

36. The method of claim 23, further comprising the administration of a single-photon emission computed tomography test to a human during said cardiac stress.

37. The method of claim 23, wherein the cardiac stress comprises pharmacologic stress.

38. The method of claim 37, wherein the pharmacologic stress is induced by the administration of adenosine to the mammal.

39. The method of claim 37, wherein the pharmacologic stress is induced by the administration of dobutamine to the mammal.

40. The method of claim 23, wherein the relative change of the marker related to BNP is at least about 10%.

41. The method of claim 23, wherein the relative change of the marker related to BNP is from about 10% to about 400%.

42. The method of claim 23, wherein the relative change in the marker related to BNP is at least about 1% per minute of exercise.

43. The method of claim 42, wherein the relative change in the marker related to BNP is at least about 5% per minute of exercise.

44. The method of claim 42, wherein the relative change in the marker related to BNP is from about 5% to about 27% per minute of exercise.
